(19) United States
(12) Patent Application Publication
(10) Pub. No.: US 2009/0068638 A1
(43) Pub. Date: Mar. 12, 2009

(54) PHAGE-BASED METHOD FOR THE DETECTION OF BACTERIA

(75) Inventors: Arghavan SHABANI, Montreal (CA); Mohammed ZOUROB, St-Laurent (CA); Beatrice ALLAIN, Montreal (CA); Rosemonde MANDEVILLE, Montreal (CA)

Correspondence Address: ROBIC, CENTRE CDP CAPITAL, 1001, VICTORIA SQUARE - BLOC E - 8TH FLOOR, MONTREAL, QC, H2Z 2B7, CA

(73) Assignee: BIOPHAGE INC., Montreal (CA)

(21) Appl. No.: 12/181,751

(22) Filed: Jul. 29, 2008

Related U.S. Application Data
(63) Continuation-in-part of application No. 11/081,687, filed on Mar. 17, 2005.

(51) Int. Cl. G01N 33/53; C12M 1/34

(52) U.S. Cl. 435/5; 435/287.1

(57) ABSTRACT

The present invention relates to the field of biosensors useful for detecting bacteria. More particularly, the present invention relates to an electrochemical cell or biosensor and its use in a phage-based method and kit for the detection of bacteria.

### CROSS-RELATED APPLICATION

This application is a continuation-in-part application of application Ser. No. 11/081,687 filed on Mar. 17, 2005.

### FIELD OF THE INVENTION

The present invention relates to the field of biosensors useful for detecting bacteria. More particularly, the present invention relates to an electrochemical cell or biosensor and its use in a phage-based method for the detection of bacteria.

### BACKGROUND OF THE INVENTION

The rapid and specific detection of pathogenic bacteria is very important for diagnosing a bacterial infection/contamination and for ensuring the safety of human health. Examples where rapid intervention through the detection of pathogenic bacteria is required include, for instance, bioterrorism attacks, contamination of water and food supplies, infection outbreaks in hospitals and in the public at large, contamination in fossil and nuclear power plants, quality of indoor/outdoor air such as the quality of the air in building ventilation and quality of indoor/outdoor water such as the water quality of pools, beaches and city water supplies (Deisingh, A. K.; Thompson, M. (2002) Analyst, 127, 567-581).

Infectious diseases caused by bacteria account for as many as 40% of the 50 million annual deaths worldwide and, more specifically in many developing countries, where microbial diseases constitute the major cause of illness and death (Mead, P. S.; Slutsker, L.; Dietz, V.; McCaig, L. F.; Bresee, J. S.; Shapiro, C. (1999) Emerg. Infect. Dis., 5, 607-625. Ivnitski, D.; Abdel-Hamid, I.; Atanasov, P.; Wilkins, E.; Stricker, S. (2000) Electroanalysis, 12, 317-325).

Conventional microbiological methods for determining bacterial cell counts include culture in selective media, biochemical, and serological characterization. Although these methods achieve sensitive and selective bacterial detection, they typically require days to weeks to yield a result. Some of the emerging technologies that have been used for the detection of bacteria include enzyme linked immunosorbent assay (ELISA), a well established pathogen detection technique (Hobson, N. S.; Tothill, I.; Turner, A. P. F. (1996) Biosensors & Bioelectronics, 11, 455-477), polymerase chain reaction (PCR) that is extremely sensitive but requires pure sample preparation and hours of processing, along with expertise in molecular biology (Higgins, J. A.; Nasarabadi, S.; Karns, J. S.; Shelton, D. R.; Cooper, M.; Gbakima, A.; Koopman, R. P. (2003) Biosensors & Bioelectronics, 18, 1115-1123), DNA hybridization (Edelstein, R. L.; Tamanaha, C. R.; Sheehan, P. E.; Miller, M. M.; Baselt, D. R.; Whitman, L. J.; Colton, R. J. (2000) Biosensors & Bioelectronics, 14, 805-813), flow cytometry which is a highly effective means for rapid analysis of individual cells at rates generally up to 1000 cells/sec, matrix-assisted laser desorption/ionization, immunomagnetic techniques, and the combination of immunomagnetic separation and flow cytometry which enabled the detection of 10³ cells/mL of E. coli O157: H7 within 1 hour (Seo, K. H.; Brackett, R. E.; Frank, J. F.; Hillard, S. (1998) Journal of Food Protection, 61, 812-816).

These detection methods are relevant for laboratory use but cannot adequately serve the needs of health practitioners and monitoring agencies in the field. Furthermore these systems are costly, require specialized training, have complicated processing steps in order to culture or extract the pathogen from the food samples, and are time consuming.

Consequently, the use of biosensors has been an important development in that they may be inexpensive, easy to use, portable, sensitive and capable of providing results in minutes. In general, biosensors are composed of a biological recognition element acting as a receptor, and a transducer which converts the ensuing biological activity into a measurable signal, commonly optical or electrical in nature (D'Souza, S. F. (2001) Biosensors & Bioelectronics, 16, 337-353). A variety of biosensors has been reported in the literature for bacterial detection including piezoelectric biosensors, electrical sensors (Edelstein, R. L.; Tamanaha, C. R.; Sheehan, P. E.; Miller, M. M.; Baselt, D. R.; Whitman, L. J.; Colton, R. J. (2000) Biosensors & Bioelectronics, 14, 805-813); Gau, J., Jr.; Lan, E. H.; Dunn, B.; Ho, C.-M.; Woo, J. C. S. (2001) Biosensors & Bioelectronics, 16, 745-755; Abdel-Hamid, I.; Ivnitski, D.; Atanasov, P.; Wilkins, E. (1999) Biosensors & Bioelectronics, 14, 309-316; Abdel-Hamid, I.; Ivnitski, D.; Atanasov, P.; Wilkins, E. (1999) Analytica Chimica Acta, 399, 99-108), surface plasmon resonance sensors (Fratamico, P. M.; Strobaugh, T. P.; Medina, M. B.; Gehring, A. G. (1998) Biotechnology Techniques, 12, 571-576; Perkins, E. A.; Squirrell, D. J. (2000) Biosensors & Bioelectronics, 14, 853-859), and optical waveguide-based devices (Zourob, M.; Mohr, S.; Brown, B. J. T.; Fielden, P. R.; McDonnell, M. B.; Goddard, N. J. (2005) Analytical Chemistry, 77, 232-242).

Electrochemical biosensors are particularly interesting because they are usually inexpensive, are well adapted to miniaturization, and can therefore provide disposable-type chips for field applications. Electrochemical sensors reported in the literature for detecting bacteria are mainly based on monitoring bacterial growth onto the transducer (Yang, L.; Ruan, C.; Li, Y. (2003) Biosensors & Bioelectronics, 19, 495-502), or the interaction between bacteria and biological recognition elements such as antibodies (Abdel-Hamid, I.; Ivnitski, D.; Atanasov, P.; Wilkins, E. (1999) Analytica Chimica Acta, 399, 99-108; Mascini, M.; Tothill, I. E.; Turner, A. P. F. (1998) Analytical Chemistry, 70, 2380-2386) and nucleic acids (DNA/RNA) (Call, D. R.; Brockman, F. J.; Chandler, D. P. (2001) International Journal of Food Microbiology, 67, 71-80; Katz, E.; Willner, I. (2003) Electroanalysis, 15, 913-947; Zhao, Y.-D.; Pang, D.-W.; Hu, S.; Wang, Z.-L.; Cheng, J.-K.; Qi, Y.-P.; Dai, H.-P.; Mao, B.-W.; Tian, Z.-Q.; Luo, J.; Lin, Z.-H. (1999) Analytica Chimica Acta, 388, 93-101; Elsholz, B.; Woerl, R.; Blohm, L.; Albers, J.; Feucht, H.; Grunwald, T.; Juergen, B.; Schweder, T.; Hintsche, R. (2006) Analytical Chemistry, 78, 4794-4802; Farabullini, F.; Lucarelli, F.; Palchetti, I.; Marrazza, G.; Mascini, M. (2007) Biosensors & Bioelectronics, 22, 1544-1549). Certain bacterial detection methods reported in the literature are mainly based on the interaction between bacteria and antibodies immobilized onto a gold surface acting as a transducer (Radke, S. M.; Alocilja, E. C. (2005) Biosensors and Bioelectronics, 20, 1662-1667). Others also present the possibility of having a dense virus layer attached to a gold electrode surface through a self-assembled monolayer (Yang, L. M. C.; Tam, P. Y.; Murray, B. J.; McIntire T. M.; Overstreet, C. M.; Weiss, G. A.; Penner, R. M. (2006) Anal. Chem., 78, 3265-3270).

Bacteriophages are small viruses which are ubiquitous in nature, highly specific to bacteria and thus harmless to humans, much cheaper to produce than antibodies and present a far longer shelf life. There are different types of bacteriophages, each capable of detecting a specific type of bacterium. For example, the T4 phage is known to bind and recognize E. Coli as its specific target.

Hence, in light of the afore-mentioned, there is a need for a phage-based method for rapid bacterial pathogen detection which, by virtue of its design and its components, would be more versatile, efficient and less costly and which would be able to overcome some of the above-discussed problems.

### SUMMARY OF THE INVENTION

In accordance with the present invention, the above object is achieved, as will be easily understood, with a phage-based method for the detection of a bacterium in a sample, such as the one briefly described herein and exemplified in the accompanying drawings.

In accordance with an aspect of the present invention, there is provided a method for detecting the presence or absence of a bacterium in a sample, the method comprising the following steps:
a) providing an electrochemical cell comprising at least one detecting electrode, at least one counter electrode and at least one phage which specifically binds said bacterium, each of said phage being covalently bound to a corresponding one of at least one detecting electrode;
b) contacting a sample suspected of containing the bacterium with the detecting electrode to create a phage-bacterium binding complex;
c) applying an electrical signal to the electrochemical cell;
d) measuring an impedance shift between the detecting electrode and the counter electrode; and
e) comparing the impedance shift obtained in step (d) with a control impedance;
   wherein a change in the impedance with respect to the control impedance is indicative of the presence of the bacterium.

Another aspect of the invention is concerned with an electrochemical cell which comprises at least one detecting electrode, at least one counter electrode and at least one phage which specifically binds said bacterium, each of said phage being covalently bound to a corresponding one of at least one detecting electrode.

Yet, another aspect of the present invention, there is provided a kit for the phage-based detection of a bacterium, the kit comprising the reagents to perform the method as defined hereinabove.

The present invention provides at least one of the following advantages, but is not limited to these:
to develop efficient and cost-effective sensor devices for the direct detection of pathogenic bacteria;
to detect both viable and non-viable bacteria;
to avoid using labelled reagents;
to detect the bacterium in real-time and simultaneously obtain qualitative and quantitative results;
to generate results more rapidly than by using pre-existing technologies;
to use phages as they are more robust, highly specific and have a long shelf life compared to antibodies;
to simultaneously allow detection of one or more different types of bacteria in a sample; and

to allow multiplexing by using multiple specific phages for different types of bacteria on multiple detecting electrodes.

The objects and advantages of the present invention will become more apparent upon reading of the following non-restrictive description of preferred embodiments thereof, given for the purpose of exemplification only, with reference to the accompanying drawings.

### BRIEF DESCRIPTION OF THE DRAWINGS

FIG. 1 is a schematic diagram for the method of the present invention in accordance with a preferred embodiment thereof.

FIGS. 2A and 2B represent a TOF-SIMS spectrum for a surface of a detecting electrode during functionalization, indicating the presence of CNO⁻ and CN⁻ fragments, according to a preferred embodiment of the present invention. FIG. 2A shows the spectrum when CNO⁻ is present and FIG. 2B shows the spectrum when CN⁻ is present. Each of FIGS. 2A and 2B show the results for the bare, 1-(3-dimethylaminopropyl) ethylcarbodiimide hydrochloride (EDC)-modified, and T4 phage immobilized surfaces of the detecting electrode.

FIGS. 3A to 3C represent 40×40 μm² intensity maps of various positive and negative ions from a surface of a detecting electrode during functionalization in accordance with a preferred embodiment of the present invention. FIG. 3A shows a bare detecting electrode. FIG. 3B shows the detecting electrode that has been modified with 0.1M EDC in 0.12N HCl. FIG. 3C shows the detecting electrode binding a T4 phage. Ion intensity is scaled individually to show maximum counts as white and zero counts in black.

FIGS. 4A and 4B show fluorescence images. FIG. 4A shows fluorescence images of T4-modified detecting electrodes at specific times following contact with a GFP-labeled E.coli.K12 sample. Magnification of 400× was the same for all four images. FIG. 4B shows fluorescence images of T4-modified detecting electrodes (arrow), compared to non-modified detecting electrodes on the same chip, following 60 minutes contact with the GFP-labeled E.coli.K12 sample (left photo shows no T4-modified electrode). Magnification of 100× was the same for the two images. All detecting electrodes (such as the one being pointed at) have a surface area of around 0.2 mm².

FIG. 5 shows scanning electron microscope (SEM) images of bacteria bound to a phage-modified surface of a detecting electrode. FIG. 5A shows T4 phage immobilized to the surface of the detecting electrode and FIG. 5B shows E. coli bacteria bound to immobilized T4 phage (high resolution). FIG. 5C shows E. coli bacteria bound to immobilized T4 phage (low resolution) FIG. 5D shows that Salmonella bacteria did not bind to T4 immobilized phages on the detecting electrode.

FIG. 6 shows variation in impedance at specific times following contact of E. coli solution with T4 modified detecting electrode which is indicative of the monitoring of cell lysis.

FIG. 7 shows Nyquist plots for T4-modified surface in presence of E. coli at different concentrations.

FIG. 8 shows a dose response curve for different E. coli bacteria concentrations, wherein Z=Δ(R_{A}+R_{B}−2σ²C_{d}).

FIG. 9 is the chemical formula describing the electrochemical attachment of nitrophenyl groups to the surface of the electrode.

FIG. 10A shows cyclic voltammograms for the functionalization carbon electrode with BF₄.N₂ (C₆H₄)—NO₂ in aqueous (curve 1 first scan, curve 2 second scan).

FIG. 10B shows cyclic voltammograms for the reduction of the nitro groups to amino groups in aqueous media, after initial functionalization in aqueous media (curve 1 first scan, curve 2 second scan).

FIG. 11 depicts the chemical reaction of the reduction of nitro groups to amino groups.

FIG. 12 shows a Nyquist diagram (Zᵢ vs Zᵣ) for the Faradic impedance measurement of an SPE electrode after electrochemical modification of 2 mM 4-nitrobenzenediazonium tetrafluoroborate and reduction to amino groups in 0.1M KCl (90:10 H₂O-EtOH) solution (Curve A). The glutaraldehyde linker is shown in curve B and phage immobilization is shown in curve C.

FIG. 13 shows cyclic voltammograms of bare, glutaraldehyde-modified and phage-modified electrode.

FIG. 14 shows the electrochemical detection of anthrax. FIG. 14A shows a Nyquist plot of impedance spectra taken in PBS solution in presence of 5 mM [Fe(CN)₆]^{3-/4} (1:1) mixture for the phage and different bacterial concentrations. FIG. 14B shows an Equivalent electrical circuit used to fit the impedance spectra.

FIG. 15 shows a dose response curve for different Bacillus anthracis concentrations.

While the invention will be described in conjunction with example embodiments, it will be understood that it is not intended to limit the scope of the invention to such embodiments. On the contrary, it is intended to cover all alternatives, modifications and equivalents, as apparent to a person skilled in the art.

### BRIEF DESCRIPTION OF THE INVENTION

1. Definitions

The definitions of the terms and the expressions provided hereinbelow are to be taken in accordance with the context of the present invention for the purposes of the description of the present invention.

The term “detecting” and any and all of its derivatives, such as, for instance “to detect”, “detection”, “detected”, refer to the discovery or perception of the existence, presence or absence of a bacterium in a sample. It will be understood by a person skilled in the art that detection is performed upon the binding of the bacteria to the phage immobilized on the detecting electrode and that viability is detected by the lysis of the bacteria by the specific phage. In the context of the present invention, it may also be understood that the detection can be qualitative and/or quantitative. As such, the presence and/or the concentration of one or advantageously one or more types of bacteria can be detected.

The term “sample” refers to a variety of sample types obtained from various origins (for instance food, water, environment, body, air, etc.), either liquid or solid, and that can be used in a diagnostic or detection assay. The sample may be a solid, liquid, gaseous or a combination thereof, but a person skilled in the art will understand that it is advantageous that the sample be dissolved in a conductive liquid media, or in a conductive aqueous solution, prior to its use in accordance with the present invention.

The term “functionalization” or “modification” and any or all of its derivatives, such as, for instance “to functionalize”, “functionalize”, “functionalized”, “functionalizing”, “to modify”, “modify”, “modified”, “modifying” are equivalent and refer to the transformation of a bio-inert material into a bioactive or biofunctional material. A person skilled in the art will know that functionalization generally occurs by generating functional groups on the surface of the detecting electrode to immobilize the phage. The term “immobilization” or any or all of its derivatives, such as, for example, “immobilize”, “immobilized”, “immobilizing” are equivalent and, in the context of the present invention, refer to permanent attachment of the phage on the surface of the detecting electrode without leaching. Of course, it is to be understood that more than one type of phage may be immobilized on the surface of the detecting electrode. As such, the detection of more than one type of bacteria may be performed, since the complex formed between the phage and the bacterium is specific. Therefore, multiplex detection of bacteria may be performed.

The term “phage” refers to as a compound that facilitates the binding of the bacterium to the detecting electrode. As will be understood by a person skilled in the art, the phage is a biological entity capable of binding or infecting a bacterium. The phage is specific to a species of bacterium, for example Escherichia, or even a strain of bacterium, for example Escherichia coli or isolates thereof, such as Escherichia coli K12. In this respect, the phage may confer specificity to the present invention by allowing the binding of certain types, species or strains of bacteria and/or preventing the binding of other types, species or strains of bacteria on the sensor surface. The phage may also be able to discriminate between two very closely related bacteria.

The term “bacterium” or “bacteria” refers to any bacteria that may be bound to a phage. A non-exhaustive list of bacteria which are detectable by the method of the invention includes, but is not limited to: Actinobacillii, Aeromonas, Archaebacteria, Agrobacteria, Aromabacter, Bacilli, Bacteriodes, Bifidobacteria, Bordetella, Borrelii, Brucella, Burkholderia, Calymmatobacteria, Campylobacter, Citrobacter, Chlamydia, Clostridium, Coccus, Coprococci, Corynebacterium, Cyanobacter, Enterobacter, Enterococci, Eubacteria, Escherichia, Helicobacter, Hemophilii, Lactobacilli, Lawsonia, Legionella, Listeria, Klebsiella, Mycobacterium, Neisserii, Pasteurella, Pneumococci, Propionibacteria, Proteus, Pseudomonas, Pyrococci, Salmonella, Serratia, Shigella, Streptococci, Staphylococci, Streoticiccys, Vibrio, Xanthomonas, and Yersinia.

With respect to the contemplated phages of the present invention, the expression “specifically binds to” refers to a phage that binds with a relatively high affinity to one or more surface proteins or polypeptides of a desired bacterium, but which does not substantially recognize and bind to surface proteins or polypeptides of another bacterium.

The term “viable” is intended to mean the capacity of a bacterium to perform its intended functions. The cellular functions may vary according to the type of cell. Cellular functions may include, for example, cellular division, cellular replication, translation, transcription, protein assembly and maturation, protein secretion, storage of compounds (e.g. proteins, lipids, etc.), responsiveness to external stimuli, migration, and the like.

The term “detecting electrode” is defined as an electrode that dominates the overall impedance of the electrochemical cell. The detecting electrode may have a width or diameter from a few millimeters down to 1 μm or smaller. The detecting electrode is the electrode on which the covalent bond with the phage occurs, and on which measurements are taken as will be further explained hereinbelow. The detecting electrode allows the qualitative and quantitative detection of bacteria as will be explained hereinbelow. It is also to be understood that multiple detecting electrodes may be used in the same electrochemical cell.

The term “counter electrode” as used herein refers to the electrode used in electrochemical cell with the detecting electrode. Generally, the “counter electrode” is also referred to as the auxiliary electrode. As such, the counter electrode is used to pass current to or from a detecting electrode. It thus effects a change in polarity, opposite to that of the detecting electrode. It may also be used so as to ensure that current does not run through a third electrode, a reference electrode for instance, in a three electrode system.

The term “voltage” as used herein is defined as the numerical value of the electrical potential across or between any two points in an electric circuit. Volts are the unit of electromotive force or electric pressure. It is the electromotive force which, if steadily applied to a circuit having a resistance of one ohm (Ω), will produce a current of one ampere. When two charges have a difference of potential the electric force that results is called electromotive force (EMF). The terms “potential”, “electromotive force” and “voltage” are used herein interchangeably. The difference in voltage can further be converted into impedance measures using the following equation:

\tilde{E}/Ĩ, wherein Z is the impedance in Ohm (Ω);

E is the voltage in Volt (V); and

I is the current in Ampere (Amp).

As used herein, the term “impedance” is defined as a measure, in ohms, of the degree to which an electric circuit resists the flow of electric current when a voltage is impressed across its terminals. Impedance may also be expressed as the ratio of the voltage impressed across a pair of terminals to the current flow between those terminals. The resistance depends upon the number of electrons that are free to become part of the current and upon the difficulty that the electrons have in moving through the circuit. As such, one will understand that impedance (Z) is the alternating current (ac) analogue of resistance (R) associated with direct current (dc) measurements. When a dc potential (V) is applied to electronic circuitry, pure resistors (R) are observed to influence the passage of current (I) as described by: V=RI. Therefore, when an ac potential (non-zero frequency) is applied, other elements such as capacitors and inductors influence the flow of current. These elements impact on the magnitude and phase of ac current as given by the complex form of Ohm's law:

\tilde{E}=ZĨ

The term “reference impedance” refers to an impedance value obtained in controlled experimental conditions. As such, for specific experimental conditions, the reference impedance can be predetermined and used in other situations using similar experimental conditions to evaluate the control impedance.

2. Biosensor of the Invention

One embodiment of the present invention relates to an electrochemical cell which comprises at least one detecting electrode, at least one counter electrode and at least one phage which specifically binds to a desired bacterium. In accordance with the present invention, the phage is covalently bound to a detecting electrode. The phage may be a natural phage, a recombinant phage, a genetically modified phage, part of a phage or phage proteins.

It will be understood that the electrochemical cell may comprise a single cell or multiple cells forming an array system or microarray for high throughput screening and detection. The electrochemical cell can also comprise one detecting electrode or more than one detecting electrodes. As such, one may appreciate that in the case where the electrochemical cell comprises one detecting electrode, one or more than one type of phage may be bound to it in order to allow the detection of one or more different types of bacteria. Alternatively, a single electrochemical cell may comprise multiple detecting electrodes, each one having a different type of phage bound to each of them.

The electrochemical cell of the invention may be listed as a phage-functionalized electrochemical cell. Indeed, and as one skilled in the art may appreciate, the detecting electrode is advantageously treated (i.e. functionalized) to allow the immobilization of the phage on the detecting electrode. Such functionalization can occur, for example, by applying a first potential to the detecting electrode or by chronoamperometry or chemical modification. The detecting electrode is then electrochemically oxidized in order to permanently immobilize the phage on the detecting electrode. For instance, if the detecting electrode is a carbon electrode, the EDC may be used to modify the oxidized carbon surface. One will also understand that in the case where more than one detecting electrode is functionalized by chronoamperometry, each detecting electrode can be functionalized individually thereby enabling a multiplexing detection system.

An important aspect of the present invention is that the phage is immobilized onto the detecting electrode by way of a covalent bond. One skilled in the art will understand that the particular choice of covalent bond is directly associated with the choice of phage to be used in order to detect a desired bacterium. For instance, and as exemplified in Example 1, if one desires to detect E. coli in a sample, the phage used may be T4 and such phage may be covalently linked to the detecting electrode by way of an amide bond. In such a case, the amide bond may be between a surface protein of the phage and the electrochemically generated carboxylic groups generated on the detecting electrode. Moreover, if one wishes to detect Bacillus anthracis, the phage may be gamma phage and such a phage may be covalently linked to the detecting electrode by way of an imine bond.

With regards to the detecting electrode, it may be made from a material chosen among the group of materials comprising for example but not limited to: carbon, silica, gold, or any other metal or conductive materials electrodes or coated metals and coated conductive material or any electrode fabricated, for example but not limited to, by electroplating, photolithography and evaporation. One skilled in the art may appreciate that if the chosen detecting electrode is made of carbon, such a detecting electrode may be advantageously a screen-printed carbon electrode (SPE).

3. Phage-Based Method and Kit for Detecting Bacteria of the Invention

According to an embodiment of the invention, the electrochemical cell of the invention is used in a method for detecting bacteria. More specifically, the electrochemical cell of the present invention is advantageously used in a “so-called” phage-based method for the direct and specific impedimetric detection of bacteria.

In this connection, the present invention concerns a method for detecting the presence or absence of a bacterium in a sample. The phage-based method of the invention comprises the following steps:
a. providing an electrochemical cell defined as above;
b. contacting a sample suspected of containing the bacterium with the detecting electrode to create a phage-bacterium binding complex;
c. applying an electrical signal to the electrochemical cell;
d. measuring an impedance shift between the detecting electrode and the counter electrode; and
e. comparing the impedance shift obtained in step (d) with a control impedance,
   wherein a change in the impedance with respect to the control impedance is indicative of the presence of the bacterium.

One skilled in the art may appreciate that since the method described hereinabove is a phage-based detecting method, it provides a specific and direct detection of the bacterium.

Once the phage-bacterium binding complex has been formed (step b), the impedemetric detection of the presence or absence of the bacterium may be performed in accordance with steps c) to e).

Comprehensively, steps c) to e) may be considered as an impedimetric measurement used to detect in a qualitative manner the presence or absence of the bacterium in the sample. Examples 1 and 2 provided hereinbelow exemplify that manner in which these impedimetric measurements are performed in accordance with the present invention.

Advantageously, after step (e), a step of quantifying the amount of bacterium detected in the sample may be performed. This quantification of the concentration of bacteria in a sample may be calculated for instance according to the amount of shift in the impedance observed.

Also advantageously, after step (e), a step of determining the viability of the bacterium detected in the sample may be performed. It is indeed a significant improvement of the present invention on the methods already known in the art to determine whether or not detected bacteria are alive or dead. Once a bacterium is detected using the immobilized phages on the electrode surface, it can remain on the electrode until the process of lysis of the bacterium is completed. If the bacterium is alive, it will be lysed and it will give another shift in the impedance. If the bacterium is dead, it will not be lysed and no additional change in impedance will occur.

Therefore, the step of determining the viability of the bacterium detected in the sample involves evaluating the change in the impedance of the detecting electrode with respect to the control impedance once a given amount of time has elapsed from the time that the phage-bacterium complex was formed on the detecting electrode. As such, one skilled in the art will appreciate that the detection of the bacterium will occur whether or not the bacterium is viable as soon as the phage has bound to the surface protein of the targeted bacterium.

For example, in the method of the invention exemplified in Example 1 hereinbelow, the shift in impedance induced by the binding of the bacteria to the phage compared to a control indicates the presence of the bacteria. The lysis of bacteria by the bound phage cause decrease in the impedance. So this invention can provides both the number of bacteria bound to the phage and the number of viable bacteria that were lysed (non viable bacteria will not be lysed by the phage as the replication system of the bacteria is no longer active).

In accordance with a further embodiment of the present invention, there is provided a kit for the phage-based detection as described above. The kit comprises at least one electrochemical cell of the invention and reagents to perform the method as defined hereinabove.

The present invention will be more readily understood by referring to the following example, which is given to illustrate the invention rather than to limit its scope.

### EXAMPLE 1

### T4 Phage-Based Method for the Detection of E. Coli

### 1) Materials

1-(3-dimethylaminopropyl) ethylcarbodiimide hydrochloride (EDC), concentrated hydrochloric acid 37%, bovine serum albumine (BSA), sodium chloride, magnesium sulfate, gelatin, Tris (hydroxymethyl) aminomethane hydrochloride (Tris-HCl buffer pH 7.5) were purchased from Sigma-Aldrich. Luria Bertani (LB) media was purchased from Quelabs (Montreal, Canada) and prepared by dissolving 25 g of LB powder into 1 L of distilled water. LB-agar medium was prepared by adding 6 g of granulated agar to 400 mL of LB media. SM buffer was prepared by mixing 5.8 g NaCl, 2.0 g MgSO₄.7H₂O, 50 mL 1M Tris-HCl pH 7.5, and 1 mL 10% (w/v) gelatin in MilliQ water. The LB medium and SM buffer were autoclaved. E. coli K12 bacteria and T4 phages were obtained from ATCC 11303 and 11303-B4 respectively. GFP-labeled E. coli K12 was obtained from Dr Roland Brousseau, (BRI, Montreal). Salmonella typhimurium DT108 bacteria were obtained from Dr Sylvain Quessy, (Faculté de Médicine Véérinaire, University of Montreal).

### 2) Electrode Microarray Preparation

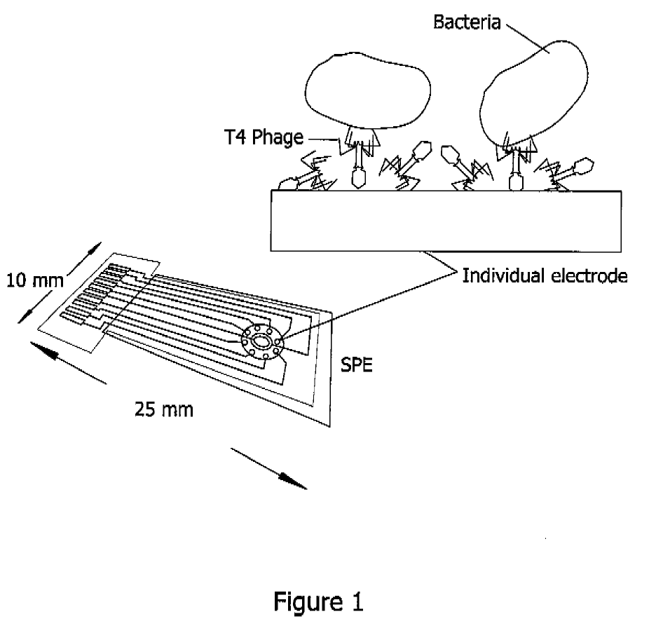

Screen printed electrodes were fabricated as previously described (Corgier, B. P.; Marquette, C. A.; Blum, L. J. (2005) J. Am. Chem. Soc., 127, 18328-18332) using graphite ink (electrodag 423 SS* (Acheson, Erstein, France) and a DEK 248* screen-printing machine (DEK, Erstein, France). The SPE platform was designed to provide multi-probe capability, easily produced by screen-printing ink onto polyester sheets. The polyester sheets produced, each carrying 16 separate electrode arrays, were subsequently baked 10 minutes at 100° C. to dry the thermoplastic carbon ink. This was followed by printing an insulating polymer (MINICO M 7000*, (Acheson, Erstein, France)) onto the microarrays, in order to define a window easily covered with a 35 μL drop of solution. This window serves to isolate the active area composed of eight 0.2 mm², individually addressable, working electrodes, one ring-shaped reference electrode, and one central auxiliary electrode (see FIG. 1).

### 3) Electrode Functionalization and Phage Immobilization

The SPEs were functionalized with 50 μL of 0.1 M 1-(3-dimethylaminopropyl) ethylcarbodiimide hydrochloride (EDC) in 0.12 N HCl, through chronoamperometry for 10 minutes. A potential of +2.2 V was applied to oxidize the carbon and generate carboxyl groups to react with the EDC (Marquette, C. A.; Lawrence, M. F.; Blum, L. J. (2006) Analytical Chemistry, 78, 959-964). The electrodes were subsequently washed thoroughly with deionized water and air dried. After the electrode functionalization, the SPEs were rinsed with deionized water and immersed in 2 mL of T4 bacteriophage solution (10⁸ pfu/mL in SM buffer solution, pH 7.5), and left on a shaker for two hours. The SPEs were subsequently washed with SM buffer (pH 7.5) several times and dipped in 2 mL BSA solution (1 mg/mL) and shaken for 40 min. Then the chips were rinsed with SM buffer for 5 min followed by covering the electrodes with 50 μL of E. coli K12 (10⁸ cfu/mL) suspension in SM buffer pH 7.5 for 20 min. After rinsing with buffer, the electrode arrays were covered with SM buffer to perform impedance measurements. Control experiments were performed by covalently immobilizing T4 phage on the electrodes and testing sensor response in the presence of SM buffer only (without bacteria) and SM buffer containing Salmonella typhimurium. To determine the limit of detection, 7-fold serial dilutions (10²-10⁸cfu/mL) of E. coli K12 were incubated over the immobilized T4 phages.

### 4) Bacteriophage and Bacteria Preparation

T4 bacteriophage (wild type) was amplified by pipetting 100 μL 10⁶ cfu/mL of E. coli K12 and 100 μL 10⁶ pfu/mL of T4 phage in a test tube and using a vortex. The mixture was incubated at room temperature for 15 min and was then added to a 20 mL tube containing LB media. The mixture was incubated for 6 hours at 37° C. in a shaking incubator. The solution was then centrifuged at 2500 g for 20 min, followed by filtering the supernatant with 0.22 μm Millex* filter (Millipore) to remove any remaining bacteria. After that the supernatant was centrifuged at high speed (12000 g) for one hour followed by removing the supernatant and resuspending the phage pellet in 1 mL of SM buffer. Phage counting was performed using soft agar plate and expressed in pfu/mL E. Coli K12 cells were grown at 37° C. in 4 mL LB media using an incubator-shaker for 3 hours, followed by centrifugation 3 times at 2500 g for 20 min, in order to exchange the media with SM buffer. Enumeration of bacteria was performed by the plate count technique and expressed in cfu/mL.

### 5) SEM Measurements

Phages immobilized on substrates were washed several times with SM buffer. Then 50 μL of host or control bacteria (10⁸ cfu/ml) were placed on the SPE surface for 15 minutes and washed with SM buffer. The images were obtained with the SEM instrument, model Hitachi S-4700* (Tokyo, Japan).

### 6) TOF-SIMS Analysis

TOF-SIMS studies were carried out with an ION-TOF SIMS IV* (Munster, Germany). The instrument has an operating pressure of 5×10⁻⁹ Torr. Samples were bombarded with a pulsed liquid metal ion source (⁶⁹Ga⁺), at an energy of 15 KeV. The gun was operated with a 27 ns pulse width and a 1.02 pA pulsed ion current for a dosage lower than 5×10¹¹ ions cm⁻², well below the threshold level of 1×10¹³ ions cm⁻² for static SIMS. Secondary ion spectra were acquired from an area of 40×40 μm, with 128×128 pixels (1 pulse per pixel), using at least 3 different positions per electrode. A chemical mapping was done on a surface of 40 μm×40 μm.

### 7) Fluorescence Measurements

The electrodes were washed several times with SM buffer after T4 phage immobilization. Then 10 μL of GFP-labeled E. coli.K12 bacteria (10⁸ cfu/mL) suspension were incubated over the immobilized phages and fluorescence images were recorded every 10 minutes up to 60 minutes to monitor the effect of phages on the bacteria. Fluorescence microscopy was performed using a Zeiss Axioplan Fluorescence Microscope* (Zeiss, Germany) equipped with a spot insight 2 megapixel color digital camera. Images were obtained using a 40× objective, using a blue filter with 450-490 nm excitation range and 515-565 nm emission range.

### 8) Impedance Measurements

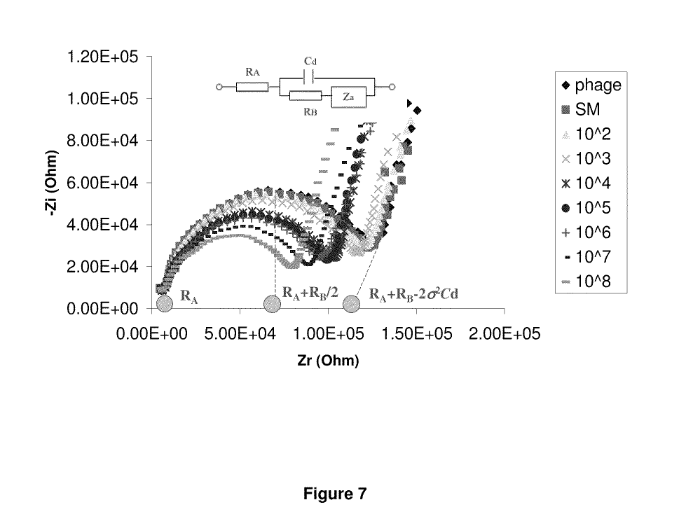

A three-carbon electrode setup was used to perform the impedance measurements. A dc potential of 400 mV, with a superimposed ac voltage of 20 mV amplitude at frequencies ranging from 100 kHz to 100 Hz, was applied to the working electrode. Results obtained under these measurement conditions showed good reproducibility (no adverse effects were observed due to the use of the centrally located ring-shaped carbon electrode of the array as pseudo-reference). All Nyquist curves were run from the high ac voltage frequency limit, to the low frequency limit (corresponding curves in FIG. 7 thus being generated from left to right). All measurements were performed in a SM buffer (pH 7.5) using a Voltalab electrochemical workstation (model PGZ 301 by Radiometer, Copenhagen). The Voltamaster* computer program (version 4.0) was used to run the electrochemical experiments and collect the data.

### 9) Results and Discussion

### 9.1) Bacteriophage Immobilization and TOF-SIMS Characterization

The electrochemical approach to functionalize the SPEs used in this work involves performing chronoamperometry in the presence of EDC, by applying a potential of +2.2 V. The outer carbon ring electrode and inner spherical electrode were used as pseudo reference and counter electrode, respectively. This oxidation of the carbon generates carboxyl groups on the surface, as described before elsewhere (Marquette, C. A.; Lawrence, M. F.; Blum, L. J. (2006) Analytical Chemistry, 78, 959-964).

More specifically, the carboxyl groups react with the EDC and produce an ester intermediate that can then react with species carrying amino groups, resulting in their covalent attachment at the surface of the electrode.

Due to the fact that the outer membrane of a phage consists of protein, they can bind to the activated carboxylic groups, resulting in the attachment of phage to the electrode through formation of amide bonds. This approach was used in this example to covalently attach T4 bacteriophage (wild type), in order to specifically detect target bacteria E. coli K12.

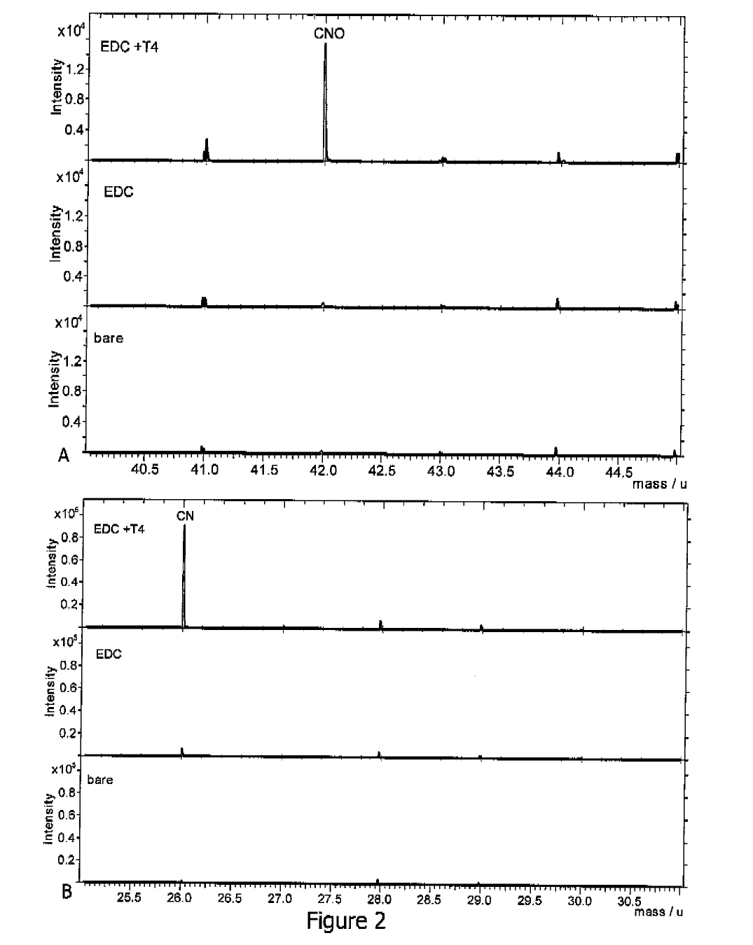

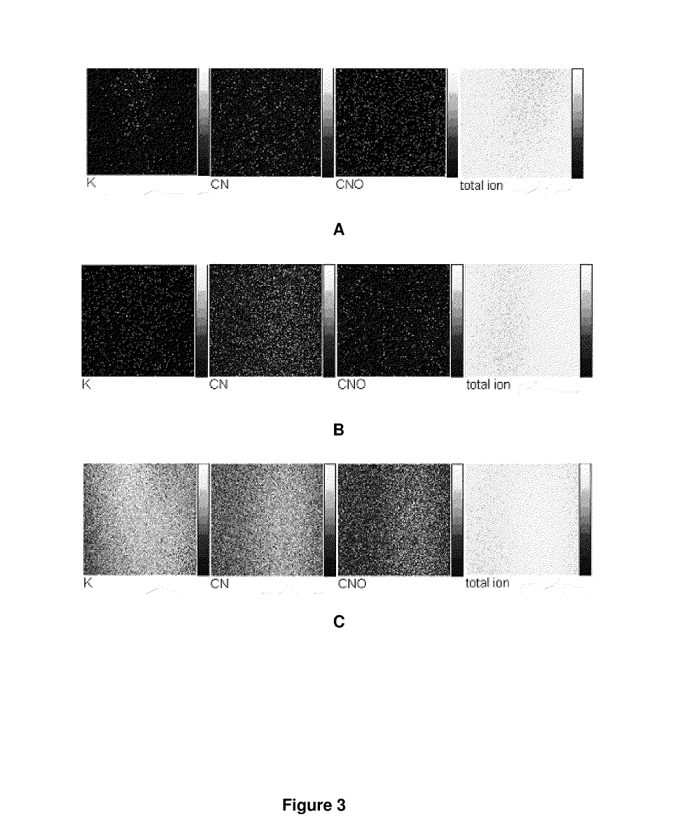

The attachment of the phage has been investigated using time-of-flight secondary ion mass spectroscopy (TOF-SIMS). The chemical mapping (secondary ion spectra) was acquired from an area of 40 μm×40 μm, using at least 3 different positions per electrode, to verify the immobilization process and confirm the attachment of phage at the electrode surface. FIG. 2 provides a useful label for monitoring the reaction since it gives rise two distinct high-intensity negative ion fragments at m/u=26 and m/u=41.9, indicating the presence of CN⁻ and CNO⁻ fragments after surface modification with EDC and the T4 immobilization. As shown in FIG. 2, the peak intensities for CN⁻ and CNO⁻ show a clear increase for a functionalized surface, compared to that of a bare electrode. This increase becomes drastically greater after phage immobilization due to the formation of amide bonds (EDC-T4, FIG. 2). FIG. 3 shows 40×40 μm² intensity maps of negative and positive fragments. It is clear from the intensity map that CN⁻ and CNO⁻ fragments are present on the EDC and T4 modified surface, showing gradually higher intensities. Also, the presence of K⁺ is a good indication of the presence of biological entities such as cells and viruses (Nygren, H.; Hagenhoff, B.; Malmberg, P.; Nilsson, M.; Richter, K. (2007) Microscopy Research and Technique, 70, 969-974), which is only observed after T4 immobilization. The relative intensity map for total ion reflects a homogenous distribution for each surface following the modification processes.

### 9.2 Fluorescence and SEM Imaging of Bacteria at T4-Modified Electrode Surfaces

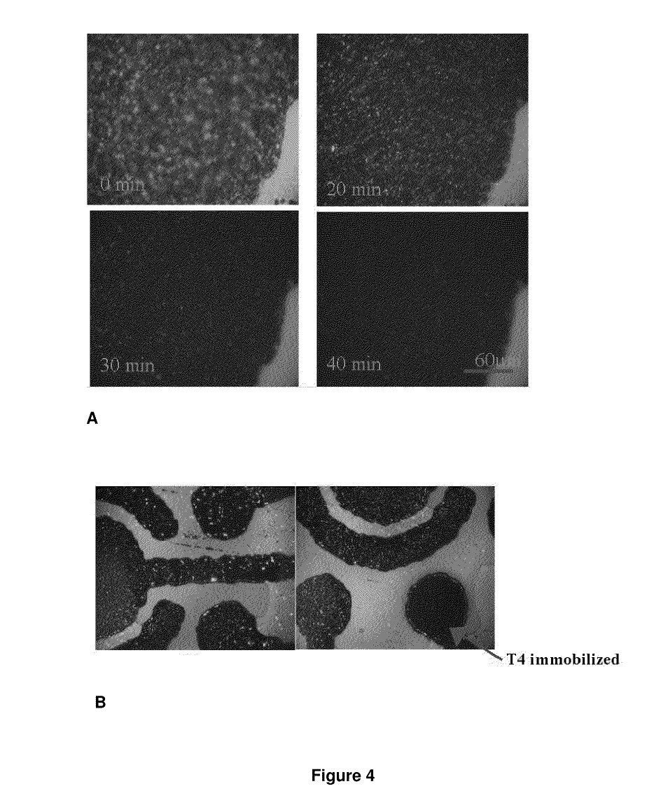

GFP-labeled E. coli.K12 was incubated with the immobilized T4 phages on the electrode surface to study the lysis effect as a function of time. The fluorescence intensity of the bacterial cells was monitored from 0 to 40 min, and the acquired fluorescence microscope images are presented in FIG. 4 A. At time zero (immediately after adding the drop of bacteria on the surface) the fluorescence intensity is maximal, and active bacteria cells can be distinguished as bright green spots. From there, the fluorescence intensity is seen to decrease with time (a distinct decrease is already visible at 20 minutes) indicating an increasing number of E. coli.K12 cells being lysed by the immobilized T4. The process is seen to be complete at 60 minutes. As a control experiment, non-functionalized electrodes (without T4) were used, and no significant decrease in the fluorescence intensity was observed, even after 60 min.

The electrochemical array approach under study presents the advantage of allowing each electrode of the same array to be addressed individually. Another control experiment was then performed by electrochemically functionalizing a single electrode of the same array, leaving the other electrodes of the array non-functionalized (without T4). GFP-labeled E. coli.K12 was incubated again on the array surface. FIG. 4B compares the fluorescence images of the non-T4 and T4-modified electrodes, on the same chip (array) after 60 minutes of bacterial incubation. FIG. 4B clearly shows that cell lysis has occurred on the T4-modified electrode (no fluorescent bacteria observed), while the non-addressed electrodes of the array (without T4) show that the GFP-labeled E. coli.K12 present at the surface are still intact after 60 minutes.

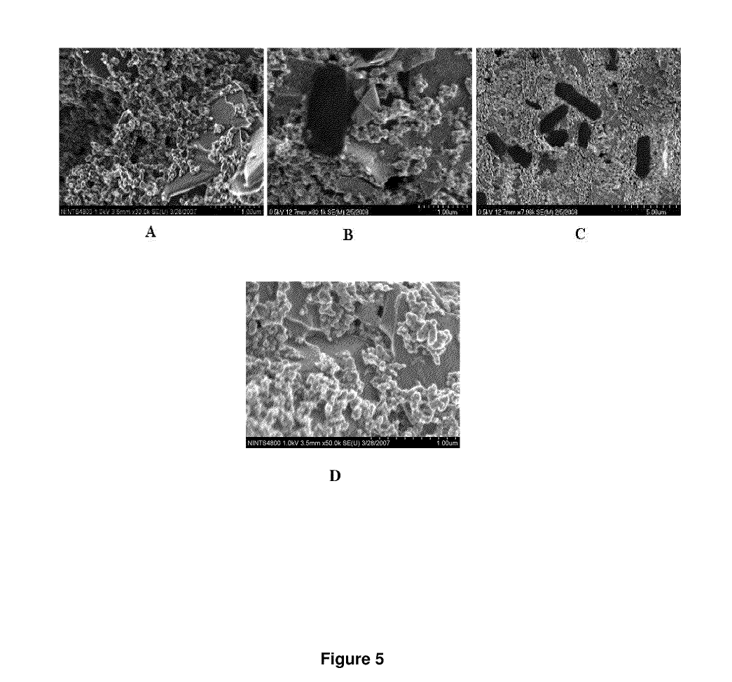

Additionally, scanning electron microscopy (SEM) was used to verify binding of bacteria to phages immobilized on the electrode surface, after rinsing in SM buffer (FIG. 5). SEM images were taken after phage immobilization (FIG. 5A) and following the binding of bacteria (FIGS. 5B and 5C). There was no distinguishable change observed in the image following phage immobilization, mainly due to the roughness of the carbon surface compared to the nanometer size of phages. After phage immobilization, 50 μL of bacteria (10⁸ cfu/mL) was incubated on the electrode surface for 10-15 min, followed by rinsing with SM buffer and acquisition of the SEM image. FIG. 5B is a higher magnification image showing one single bacteria captured by immobilized phage, and FIG. 5C is a lower magnification image showing a number of bacteria on the same electrode surface. No bacteria were observed to bind to the immobilized T4 phages on the sensor surface when Salmonella was used as a control experiment.

9.3) E. coli Detection by Electrochemical Impedance Spectroscopy (EIS)

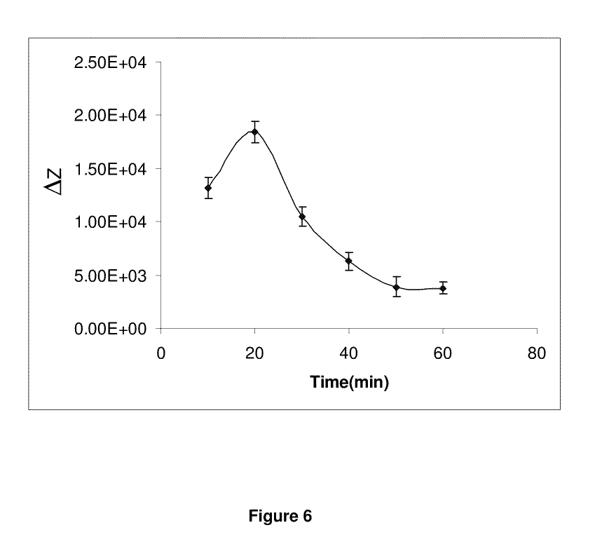

The fluorescence imaging data presented in FIG. 4 indicate that the immobilized phages effectively lyse the bacteria within a period of approximately 40 to 60 minutes. As an initial experiment, this time dependent behavior was studied with the electrochemical impedance detection approach, using T4 modified SPEs. After phage immobilization, the electrodes were washed with buffer solution and covered with 50 μL of 10⁸ cfu/mL of E. coli cells, and the shifts in impedance were recorded at different times following the incubation of the bacteria suspension. FIG. 6 shows the shifts in impedance observed from 10 to 60 min following deposition of the bacteria solution onto the electrode surface. First measurements were taken at 10 min to insure proper equilibration of the sensor device (for thermal equilibration and settling of the bacteria at the electrode surface). The results show an initial increase in impedance shift, attributed to the arrival of intact bacteria at the phage modified electrode surface, which reaches a maximum value of ˜1.9×10⁴ Ohms at 20 min. FIG. 6 also shows that the rate of shift gradually decreases after 20 min, providing an indication that the infection of the E. coli and the lytic cycle starts to occur within approximately at 20 min at 37° C. (approximately 35 min at room temperature), and levels off after 50 min.

The fluorescence observations tend to confirm the time-response for bacterial decay, as monitored by electrochemical impedance. The images presented in FIG. 4A show a progressive decrease in fluorescence intensity due to intact cells after 20 min, and indicate that most of the bacterial cells have lysed at 60 min.

FIG. 7 shows the impedance results obtained when bacteria suspensions with different concentrations (10² to 10⁸ cfu/mL) were placed on the bacteriophage-modified surface. To ensure that a maximum impedance signal was measured, the Nyquist plots were taken at 25 min of incubation with the bacteria (after lysis has begun according to FIGS. 4A and 6), with each measurement (complete curve) taking 3 min to acquire. The equivalent circuit typically used to interpret the impedance results observed with this system is also shown in FIG. 7. It consists of the resistance of the electrolyte (R_{A}), the charge transfer resistance (R_{B}), the double layer capacitance (C_{d}) and the impedance due to mass transfer (Zₐ). In the high frequency domain, the Nyquist plots are expressed by the following equation ⁶⁰:

(zᵣ−R_{A}−R_{B}/2)²+Zᵢ²=(R_{B}/2)² (1)

corresponding to a half-circle plot starting at R_{A} and having a radius value equal to R_{B}/2.

In the low frequency domain, the plots show a straight line given by the following equation:

Zᵢ=Zᵣ−R_{A}−R_{B}+2σ²C_{d} (2)

σ is a diffusion-dependent term which is inversely proportional to the concentration of electro active species in solution near the electrode surface (Bard, A. J.; Faulkner, L. R. (2001) Electrochemical Methods: Fundamentals and Applications; Wiley: New York). The numerical values of the equivalent circuit components were thus extracted from the data shown in FIG. 7, and are summarized in Table 1.

Table 1 shows that R_{A} increases by approximately 290 Ohms for E. Coli concentrations ranging from 10² to 10⁸ cfu/mL, which is basically a consequence of an increasing introduction of non-conducting bacteria (intact bacteria having insulating membranes) in the electrolyte solution. Interestingly, the results clearly indicate that the semicircle diameter, which relates directly to the value of the charge transfer resistance (R_{B}), undergoes a decrease with increasing bacteria concentration. This effect is contrary to what is usually observed for simple attachment of intact bacteria cells to an electrode (i.e. an increase of charge transfer resistance, of impedance, with increasing concentration of intact bacteria). This can be readily attributed to the fact that these measurements are being performed during lysis of the bacteria, after surface attachment. Lysis involves the breaking up of the bacterial cells and the release of highly mobile ionic material (such as K⁺ and Na⁺), thus increasing the conductivity of the media near the electrode surface. Correspondingly, the values related to charge transfer resistance (R_{B}) show a clear decrease with increasing concentration of E. Coli cells.

The effect of increasing bacteria concentration on the double layer capacitance and diffusion controlled processes at the surface, expressed by the 2σ²C_{d} values obtained from the straight line portions of the curves shown in FIG. 7, is also reported in Table 1. It should be noted that although the degree of roughness of the screen-printed carbon surfaces used in this study may preclude the formation/consideration of a double layer as described by strict theoretical formalism, the Nyquist plots however do show very good compliance with the behavior prescribed by the equivalent circuit (with C_{d} and Zₐ) shown in FIG. 7. The values for the 2σ² C_{d} factor appear to follow an initial decrease from 0 to 10⁴ cfu/mL, followed by an increase (except for the result at 10⁶ cfu/mL) at higher concentration. Although this factor has much less influence than R_{B} on the overall variation in impedance, the trend can also be attributed to the lysis of bacteria at the surface. On the one hand an increase in the concentration of ionic species at the electrode surface is reflected by a decrease in the diffusion-dependent component σ. On the other hand, it increases the dielectric permittivity and decreases the thickness of the double layer, resulting in an expected gradual increase in C_{d}, hence a decrease in impedance.

The overall effect on the variation in impedance is given in the last column of Table 1 which reports the values of Zᵣ=R_{A}+R_{B}−2σC_{d}. No change in these values was observed for a concentration of 10 cfu/mL (compared to 0 cfu/mL), and therefore a concentration of 10 cfu/mL could not be detected by this system. It should be noted, however, that very small aliquots (50 μL) were used in these studies, which corresponds to very low amounts of detected bacteria.

### 9.4) Dose Response

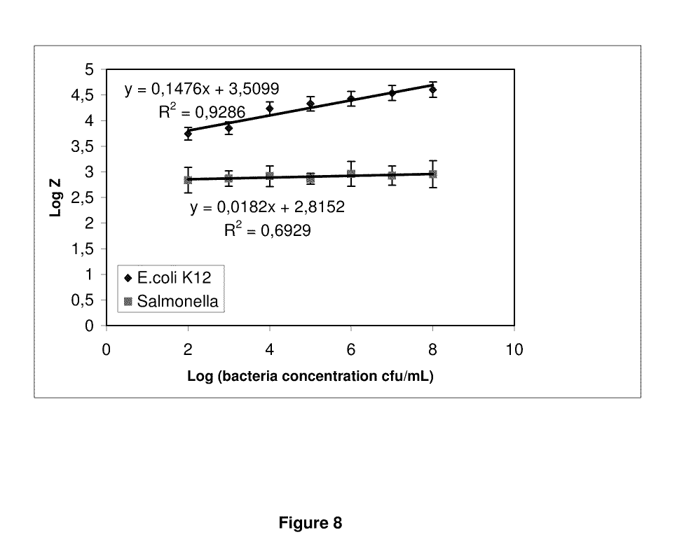

Control experiments in dose response were also performed with buffer solution only, and the non-target bacteria Salmonella, and no significant impedance shifts were observed. FIG. 8 shows a log-log plot for the impedance shifts (given as Z, corresponding to Δ(R_{A}+R_{B}−2σ² C_{d}) between curves in FIG. 7), observed as a function of E. coli and Salmonella typhimurium concentrations ranging from 10² to 10⁸ cfu/mL. For the specific target bacteria E. coli, the dose response was found to be nearly linear over seven decades of bacterial concentration, using three replicates of eight assays. The detection limit was found to be 2×10⁴ cfu/mL when 50 μL of the bacteria sample was incubated for 25 min with the immobilized T4 phages. The detection limit has been calculated from the slope of the calibration curve according the following equation:

D.L.=kσ/m (3)

Where k=3, σ=noise of blank, and m=slope of calibration curve. The same detection experiments preformed with samples containing non-specific salmonella typhimurium show comparatively minor variation over the same concentration range.

The detection approach described herein has been shown to be fast and efficient in comparison with other phage-based methods reported for bacterial detection. For example, the methods described by Goodridge et al. (Goodridge, L.; Chen, J.; Griffiths, M. (1999) Applied and Environmental Microbiology, 65, 1397-1404; Goodridge, L.; Chen, J.; Griffiths, M. (1999) International Journal of Food Microbiology, 47, 43-50), or Van Poucke and Nelis (Van Poucke, S. O.; Nelis, H. J. (2000) Journal of Microbiological Methods, 42, 233-244), are either time consuming (requiring 9-10 hours) or require fluorescence labelling. Another approach published for identification of E. coli 0157:H7, using the phage PPO1, requires genetic modification of the phage genome (Oda, M.; Morita, M.; Unno, H.; Tanji, Y. (2004) Applied and Environmental Microbiology, 70, 527-534).

### 10) Conclusion

Low-cost, versatile, and robust screen-printed carbon electrode arrays have been used as the base transducers to successfully immobilize the lytic phage, T4, to act as a recognition element for the detection of E. coli.K12 cells. The impedance measurements performed with these arrays have been shown to provide a rapid, direct and label-free means of detecting specific bacteria using a simple phage-based approach. The Nyquist plots show significant changes in the high frequency range, corresponding mainly to a decrease in charge-transfer resistance due to lysis of E. coli by T4 at the electrode surface. TOF-SIMS analysis provides solid support for the successful immobilization of the bacteriophage, and fluorescence microscopy also indicates that specific bacteria lysis is occurring only at functionalized addressable electrodes of choice. Finally, comparison of the observed impedance response in the presence of non-specific Salmonella typhimurium demonstrates this approaches potential for direct and specific detection of bacteria.

### EXAMPLE 2

### Phage-Based Method for the Detection of Anthrax

### 1) Materials

4-Nitrobenzenediazonium tetrafluoroborate, concentrated sulphuric acid, glycine, 25% glutaraldehyde, potassium chloride, phosphate buffer saline, and potassium hexaferricyanide/potassium hexaferrocyanide were purchased from Sigma-Aldrich. Bacillus anthracis and gamma phages were obtained from Felix d'Herelle collection #1140 and 388 respectively.

2) Surface Modification Using Cyclic Voltammetry

Cyclic voltammetry was performed using 2 mM 4-nitrobenzenediazonium tetrafluoroborate solution in 0.1 M H₂SO₄ in aqueous media. The nitro groups were then reduced to amino groups in 0.1M KCl (90:10 H₂O—EtOH) solution using cyclic voltammetry. For all cyclic voltammetric scans, the potential range was varied from 0.4V to −1.7 V, at a scan rate of 200 mV/sec. All cyclic voltammograms were obtained with a computer-controlled Voltalab electrochemical workstation (model PGZ 301 by Radiometer, Copenhagen). After the electrochemical modification, the electrochemical cells were rinsed with distilled-deionized water and dried under a flow of air.

### (2) Glutaraldehyde Treatment of the Electrode

The electrochemical cells were subsequently functionalized with 50 μL of 25% glutaraldehyde solution for 30 minutes prior to immobilization of the bacteriophage probes. The glutaraldehyde acts as a linker to attach the phage to the surface of the detecting electrode.

### (3) Procedure for Phage Immobilization

After treatment with glutaraldehyde, the chips were rinsed with distilled-deionized water and covered with 2 mL of bacteriophage gamma (108 pfu/mL in SM buffer solution pH=7.4) for two hours. The modified chips were then treated with glycin (dipped in 0.1 M aqueous glycine solution for 20 min) to cap off any unreacted aldehyde groups remaining after phage immobilization.

### (4) Electrochemical Measurements

A three-carbon electrode setup was used to perform the electrochemical measurements. Cyclic voltoammetry and impedance measurements were performed in 40 μL of a pH=7.4 PBS containing 8 g of NaCl, 0.2 g of KCl, 1.44 g of Na₂HPO₄, 0.24 g of KH₂PO₄, in the presence of 5 mM potassium hexaferricyanide/potassium hexaferrocyanide (1:1) mixture as a redox couple. Impedance was measured at a dc potential of 0 mV, with a superimposed ac voltage of 20 mV amplitude, at frequencies ranging from 100 kHz to 100 Hz, applied to the working electrode. Results obtained under these measurement conditions showed good reproducibility (no adverse effects were observed due to the use of the centrally located ring-shaped carbon electrode of the array as pseudo-reference).

All Nyquist curves were run from the high ac voltage frequency limit, to the low frequency limit. All measurements were performed with a Voltalab electrochemical workstation (model PGZ 301 by Radiometer, Copenhagen). The Voltamaster computer program (version 4.0) was used to calculate out-of-phase impedance (Zᵢ) and in-phase impedance (Zᵣ).

(5) Results And Discussion

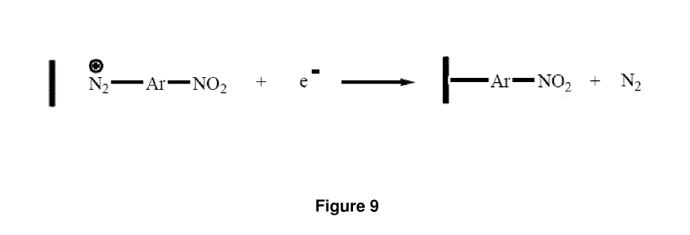

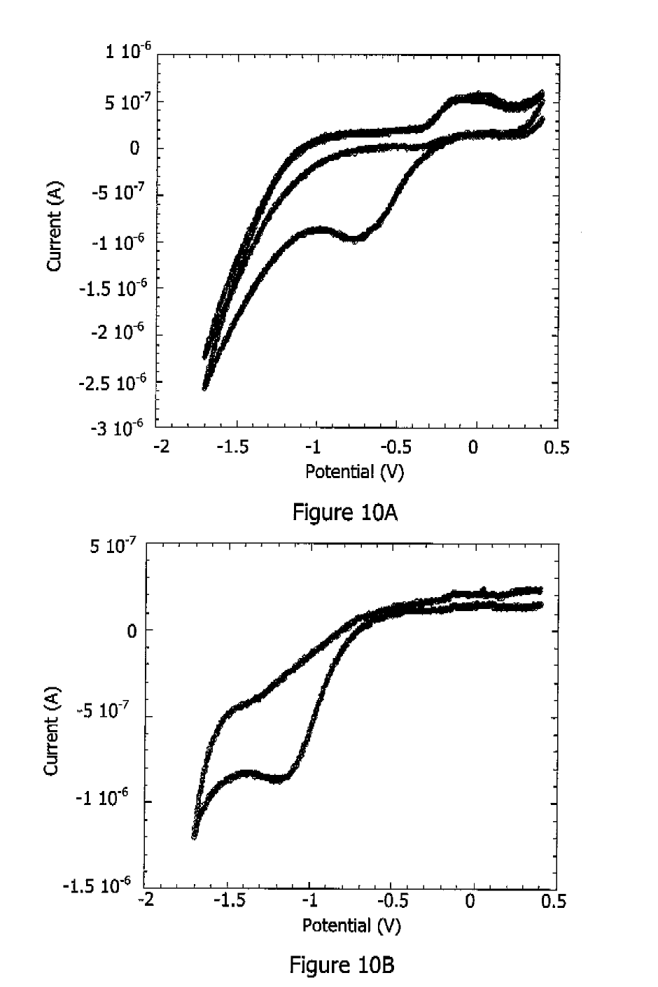

The first step for chemical functionalization of carbon electrochemical cells was carried out using cyclic voltammetry in contact with an aqueous 2 mM tetrafluoroborate 4-nitrobenzene diazonium solution in 0.1 M H₂SO₄. The initial scan (FIG. 10A, curve 1) shows a broad and irreversible cathodic wave at around −0.5 V vs carbon, which upon subsequent scans gradually tends toward a flat, near zero cathodic current profile (FIG. 10A, curve 2) due to surface passivation. This reduction process of the diazonium moiety is illustrated in FIG. 9.

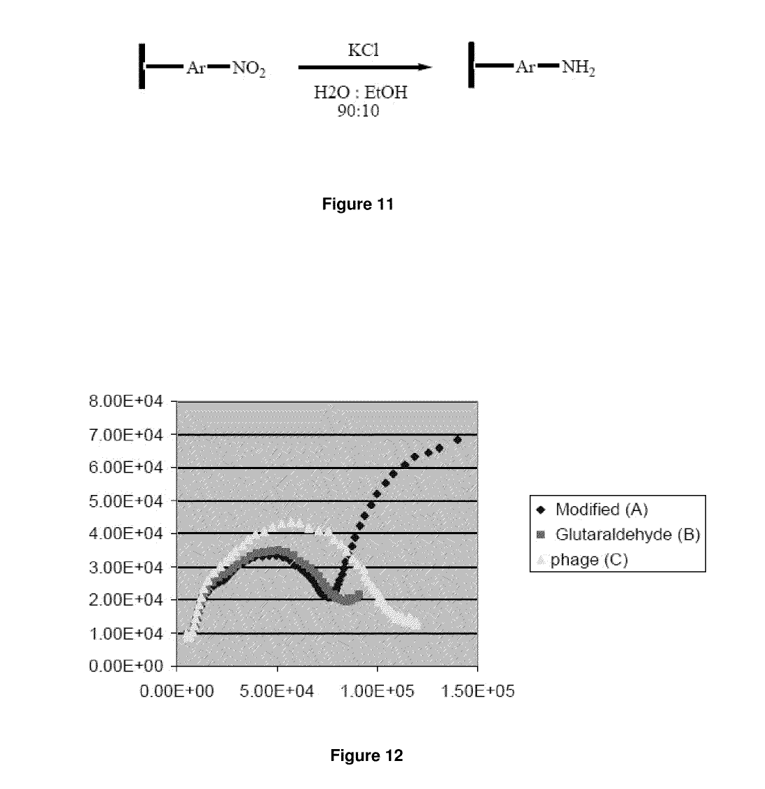

The next step in surface functionalization is the cyclic voltammetric reduction of nitro groups to amino groups using a 0.1 M KCl, (90:10 H₂O-EtOH) solution (FIG. 10B). The reduction of the nitro groups is a six-electron reduction process, as described in FIG. 11. It should be noted however, that although these voltammetric steps are intended to accomplish an overall 6 e-reduction of NO₂ to NH₂, there have been reports focused on glassy carbon surface modifications indicating that this reduction is only partial, resulting in a small fraction of NO₂ groups undergoing a 4e-reduction to form NHOH rather than NH₂.

### (6) Bacteriophage Immobilization and Characterization

Bacteriophage gamma (γ) were immobilized onto carbon electrodes (SPEs) through glutaraldehyde linker. FIG. 12 shows the faradic impedance spectra, illustrated as Nyquist plot of the modified SPE (curve A) and glutaraldehyde linker (curve B) and after phage immobilization (curve C) in a pH=7.4 PBS (1×) in the presence of potassium hexaferricyanide/potassium hexaferrocyanide (1:1) mixture as a redox couple. It can be seen from FIG. 12 that the electron transfer resistance (Rct, the diameter of semicircles) of [Fe(CN)₆]^{3-/4-} increased by small value after glutaraldehyde binding onto SPE modified surfaces but the phage immobilization resulted in a significant change in the electron transfer resistance.

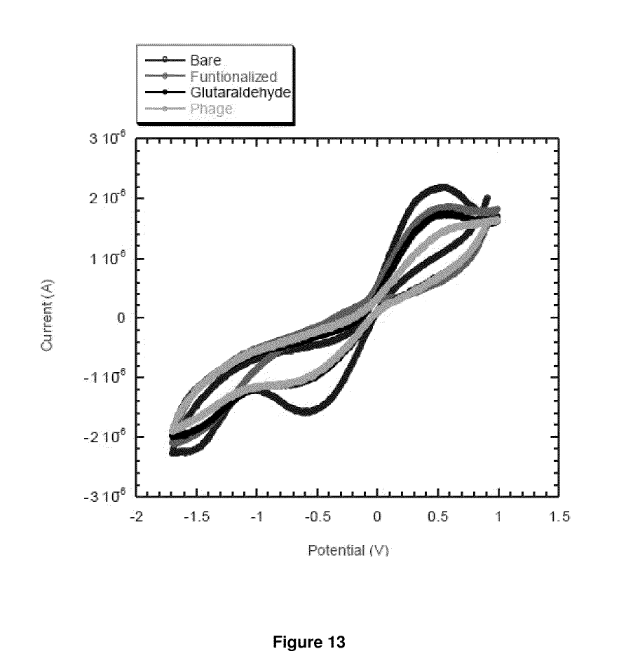

The surface modification and phage immobilization were also investigated by cyclic voltammetry using [Fe(CN)₆]^{3-/4-} as a redox probe. FIG. 13 shows the cyclic voltammograms of [Fe(CN)₆]^{3-/4-} for SPE electrodes: bare, modified, glutaraldehyde and phage electrodes, respectively. FIG. 13 shows the electron transfer between the redox probe and the electrode was decreased after each step. The blocking of the interfacial electron transfer between the soluble redox probe and the electrode upon binging phage (protein) to the surface is efficient. However, phage bound to the surface could only partly inhibit the electrical contact between [Fe(CN)₆]^{3-/4-} and the electrode surface.

### (7) Anthrax Detection Using EIS

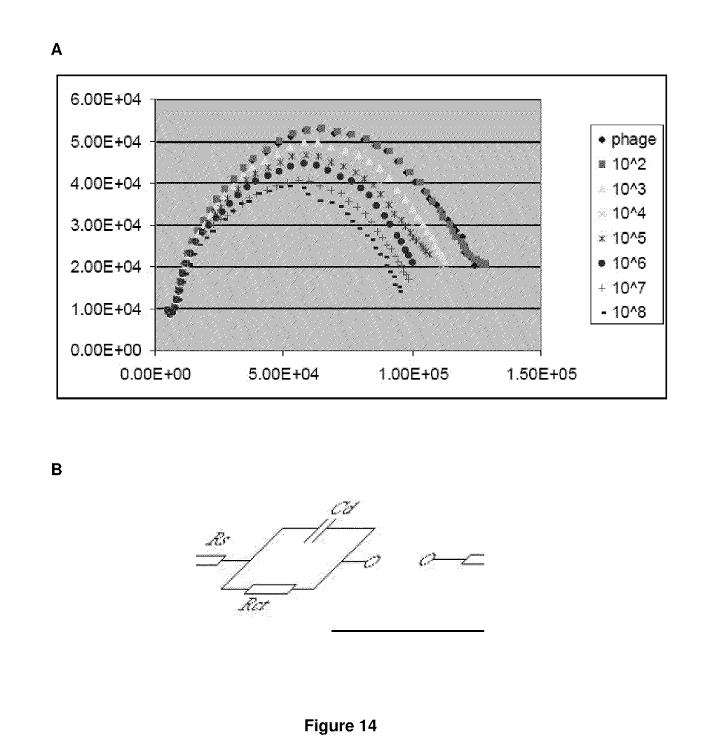

FIG. 14 shows the impedance results obtained when bacteria suspensions with different concentrations (10² to 10⁸ cfu/mL) were placed on the bacteriophage-modified surface. The impedance spectra were taken in PBS solution in presence of 5 mM[Fe(CN)6]^{3-/4-(}1:1) mixture upon treatment of sensing surface with different concentration of anthrax spores. To insure that a maximum impedance signal was measured, the Nyquist plots were taken at 40 min of incubation with the bacteria (after lysis has begun) with each measurement (complete curve) taking 3 min to acquire.

The equivalent circuit typically used to interpret the impedance results observed with this system is also shown in FIG. 14. It consists of the resistance of the electrolyte (Rs), the charge transfer resistance (Rct), the double layer capacitance (Cd). Interestingly, the results clearly indicate that the semicircle diameter, which relates directly to the value of the charge transfer resistance (Rct), undergoes a decrease with increasing bacteria concentration. This effect is contrary to what is usually observed for simple attachment of intact bacteria cells to an electrode (i.e. an increase of charge transfer resistance, of impedance, with increasing concentration of intact bacteria). This can be readily attributed to the fact that these measurements are being performed during lysis of the bacteria, after surface attachment. Lysis involves the breakup of the bacterial cells and the release of highly mobile ionic material (such as K+and Na+), thus increasing the conductivity of the media near the electrode surface.

FIG. 14 shows the electrochemical detection of anthrax. FIG. 14A shows a Nyquist plot of impedance spectra taken in PBS solution in presence of 5 mM [Fe(CN)6]3−/4−(1:1) mixture for the phage and different bacteria concentration. FIG. 14B shows an Equivalent electrical circuit used to fit the impedance spectra.

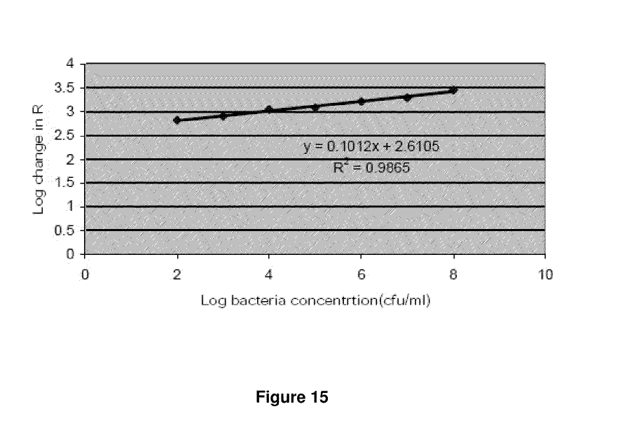

FIG. 15 shows a log-log plot for the impedance shifts. The changes in resistance are calculated following equation: ΔR=Rb−Rp, where Rp is the value of resistance when the phage immobilized on the electrode and Rb is the value of resistance after adding bacteria.

### Conclusion

Carbon surface were functionalized by a simple 2-step cyclic voltammetric reduction process using a diazonium salt as starting compound, and then treated with glutaraldehyde to act as a linker for the attachment of the bacteriophage gamma. Faradic impedance spectra and cyclic voltametric were taken to provide evidence for binding the phage to the modified surfaces. This surface probe was used to detect anthrax spores ranging from 10²-10⁸ cfu/ml. The Nyquist plots changed in the high frequency ranges, corresponding to decrease in charge transfer resistance due to lysis of anthrax at the electrode surface.

Although a preferred embodiment of the present invention has been described in detail herein and illustrated in the accompanying drawings, it is to be understood that the invention is not limited to this embodiment and that various changes and modifications could be made without departing form the scope and spirit of the present invention.

TABLE 1

| E.coli | | | | |
|---|---|---|---|---|
| concentration | R_{A} | R_{B} | | |
| (cfu/mL) | (kΩ)) | (kΩ) | 2σ²Cd (kΩ) | (R_{A} + R_{B} −2σ²Cd) (kΩ) |
| 0 | 5.67 | 121.22 | 15.13 | 111.76 |
| 10² | 5.70 | 117.71 | 12.75 | 110.66 |
| 10³ | 5.75 | 113.99 | 10.69 | 109.05 |
| 10⁴ | 5.88 | 102.39 | 9.32 | 98.95 |
| 10⁵ | 5.85 | 102.57 | 13.55 | 94.871 |
| 10⁶ | 5.83 | 91.93 | 8.36 | 89.39 |
| 10⁷ | 5.94 | 89.64 | 13.78 | 81.80 |
| 10⁸ | 5.96 | 88.28 | 18.30 | 75.94 |

1. A method for detecting the presence or absence of a bacterium in a sample, the method comprising the following steps:
a) providing an electrochemical cell comprising at least one detecting electrode, at least one counter electrode and at least one phage which specifically binds said bacterium, each of said phage being covalently bound to a corresponding one of at least one detecting electrode;
b) contacting a sample suspected of containing the bacterium with the detecting electrode to create a phage-bacterium binding complex;
c) applying an electrical signal to the electrochemical cell;
d) measuring an impedance shift between the detecting electrode and the counter electrode; and
e) comparing the impedance shift obtained in step (d) with a control impedance;
wherein a change in the impedance with respect to the control impedance is indicative of the presence of the bacterium.

2. The method of claim 1, comprising, after step (e), a step of quantifying the amount of bacterium detected in the sample.

3. The method of claim 1, comprising, after step (e), a step of determining the viability of the bacterium detected in the sample.

4. The method of claim 1, wherein the detecting electrode is made from a material chosen among the group of materials comprising: carbon, silica, gold, other metal or conductive materials, electrodes or coated metals, and coated conductive materials.

5. The method of claim 4, wherein the detecting electrode is made of carbon.

6. The method of claim 5, wherein the detecting electrode is a screen-printed carbon electrode (SPE).

7. The method of claim 1, wherein the electrochemical cell is a single cell or an array of electrochemical cells with single or multiple detecting electrodes in each cell.

8. The method of claim 1, wherein the phage is a natural phage, a recombinant phage, a genetically modified phage, part of a phage or phage proteins.

9. The method of claim 1, wherein the bacterium is chosen from the group consisting of: Actinobacillii, Aeromonas, Archaebacteria, Agrobacteria, Aromabacter, Bacilli, Bacteriodes, Bifidobacteria, Bordetella, Borrelii, Brucella, Burkholderia, Calymmatobacteria, Campylobacter, Citrobacter, Chlamydia, Clostridium, Coccus, Coprococci, Corynebacterium, Cyanobacter, Enterobacter, Enterococci, Eubacteria, Escherichia, Helicobacter, Hemophilii, Lactobacilli, Lawsonia, Legionella, Listeria, Klebsiella, Mycobacterium, Neisserii, Pasteurella, Pneumococci, Propionibacteria, Proteus, Pseudomonas, Pyrococci, Salmonella, Serratia, Shigella, Streptococci, Staphylococci, Streoticiccys, Vibrio, Xanthomonas, and Yersinia.

10. An electrochemical cell which comprises at least one detecting electrode, at least one counter electrode and at least one phage which specifically binds said bacterium, each of said phage being covalently bound to a corresponding one of at least one detecting electrode.

11. A kit for the phage-based detection of a bacterium, the kit comprising an electrochemical cell as defined in claim 10 and reagents to perform the method as defined in claim 1.
